(19) United States
(12) Patent Application Publication
(10) Pub. No.: US 2025/0186570 A1
(43) Pub. Date: Jun. 12, 2025

(54) ANTHRAX VACCINE

(71) Applicant: Pelican Technology Holdings, Inc., San Diego, CA (US)

(72) Inventors: Hubert CHEN, San Diego, CA (US); Jane C. SCHNEIDER, San Diego, CA (US)

(21) Appl. No.: 18/523,662

(22) PCT Filed: Jun. 3, 2022
(86) PCT No.: PCT/US2022/032212

§ 371 Date: Nov. 29, 2023

Related U.S. Application Data
(60) Provisional application No. 63/209,284, filed on Jun. 10, 2021.

(51) Int. Cl. A61K 39/07; A61K 9/1273; A61K 39/00; A61P 31/04; A61P 37/04

(52) U.S. Cl. CPC A61K 39/07; A61K 9/1273; A61P 31/04; A61P 37/04; A61K 2039/545; A61K 2039/55555; A61K 2039/55572; A61K 2039/575

(57) ABSTRACT

Provided herein is an immunogenic composition, comprising: 5 mcg to 100 mcg B. anthracis rPA (recombinant protective antigen) or mrPA (mutant rPA) protein and liposome-embedded MPLA (monophosphoryl lipid A) adjuvant. An immunogenic composition comprising 60 mcg to 600 mcg B. anthracis mrPA (mutant rPA) protein, wherein the composition is free of adjuvant, i.e., with no added adjuvant, is also provided. Also described is a method for inducing an immune response to B. anthracis, the method comprising administering the immunogenic composition to the subject.

### CROSS-REFERENCING

This application claims the benefit of U.S. provisional application Ser. No. 63/209,284, filed on Jun. 10, 2021, which application is incorporated by reference herein for all purposes.

### STATEMENT REGARDING FEDERALLY SPONSORED RESEARCH OR DEVELOPMENT

This invention was made with Government support under contract HHSO100201500011C awarded by the Department of Health and Human Services; Office of the Assistant Secretary for Preparedness and Response; Biomedical Advanced Research and Development Authority. The Government has certain rights in the invention.

### BACKGROUND

Bacillus anthracis is a Tier-1 biothreat agent that poses a great risk to United States' national security and public health due to its exceptionally high virulence. Bacillus anthracis is the causative agent of anthrax, one of the deadliest bioterror agents. Anthrax causes rapid death, in 3-6 days, of 85-100% of exposed individuals, unless antibiotics are administered within 20-24 hours after the onset of symptoms. Intentional release of these organisms as a bioweapon could lead to massive deaths, public panic, and social chaos. The best way to offset such an attack is to vaccinate people prior to the attack. Vaccination is also essential after the attack to minimize further casualties and to mitigate additional attacks. Consequently, since the anthrax attacks of September 2001, stockpiling of vaccines against anthrax has been a national priority.

The BIOTHRAX® vaccine (or anthrax vaccine alum-adsorbed; AVA) was approved in 1970s, and has been used for high risk individuals such as the military. BioThrax is approved for pre-exposure prophylaxis of disease in persons at high risk of exposure and post-exposure prophylaxis of disease following suspected or confirmed Bacillus anthracis exposure, when administered in conjunction with recommended antibacterial drugs. This vaccine consists of a filtered crude culture supernatant of B. anthracis strain V770-NP1-R, but it exhibits significant reactogenicity in vaccinated individuals. A re-formulated version of the BIOTHRAX vaccine (AV7909, Emergent BioSolutions, Gaithersburg, Md.) is in development for use in humans (18-65 years of age) to prevent disease following suspected or confirmed exposure to B. anthracis in conjunction with recommended antibiotic treatment(s). The use of this reformulated vaccine is currently limited to military and high-risk healthcare workers. The reformulated vaccine requires multiple boosters to maintain protective immunity and it is believed to generate significant adverse events.

This disclosure provides a new, efficient vaccine against anthrax that has the potential to overcome the limitations of the incumbent technology.

### SUMMARY

Provided herein is an immunogenic composition comprising: (a) 5 mcg-100 mcg B. anthracis rPA (recombinant protective antigen) or mrPA (mutant rPA) protein and (b) liposome-embedded MPLA (monophosphoryl lipid A) adjuvant. An immunogenic composition comprising 60 mcg to 600 mcg B. anthracis mrPA (mutant rPA) protein, wherein the composition is free of adjuvant, i.e., with no added adjuvant, is also provided. Also provided is a method for inducing an immune response to B. anthracis, the method comprising administering the immunogenic composition to the subject. The composition may be used as a vaccine against anthrax.

Further details of the composition and method are described below.

### BRIEF DESCRIPTION OF THE FIGURES

FIG. 1. Study design and subject allocation ratio. The timing of doses (hollow arrow) and assessment of safety and immunogenicity (solid arrow) of 10, 50, and 80 mcg doses of mrPA with MPLA-liposome adjuvant (Px563L), 10, 50, and 80 mcg doses of mrPA without adjuvant (RPA563) or placebo is shown. The number of subjects allocated to each arm is shown for each cohort.

FIG. 2. Injection site reactions. The percentage of subjects with grade 1 (solid fill) or grade 2 (no fill) injection site reactions through Study Day 70 are shown.

FIG. 3. Systemic reactogenicity. The percentage of subjects with grade 1 (solid fill) systemic reactogenicity through Study Day 70 are shown.

FIGS. 4A and 4B. Immunogenicity profiles of Px563L and RPA563 through Study Day 70. The geometric mean titer±95% confidence intervals is shown for 10 mcg mrPA (♦), 50 mcg mrPA (▴), 80 mcg mrPA (▪), and placebo (●). Horizontal dashed line (red) depicts TNA NF50 value of 0.56 for reference. (FIG. 4A) Px563L (FIG. 4B) RPA563.

FIG. 5. TNA NF₅₀ and Survival. Toxin Neutralization Assay geometric mean NF50 values of all rabbits and survival within 14 days subsequent to a lethal dose of B. anthracis spores, which were administered on Day 70.

FIG. 6. Anti-PA antibodies produced by animals injected (IM) with 80 mcg of RPA563 (Group 3) on days 1, 14 and 28 through study day 42; n=7 animals per sex for all timepoints except Day 42 (n=2 per sex). Upper panel shows mean titer (units/mL) for male (●) and female (▪) rabbits, with dashed line indicating that only 2 animals were evaluated at Day 42. The bottom panel shows anti-PA titer for each individual animal in the study for each timpoint sampled.

FIG. 7. Anti-PA antibodies produced by animals injected (IM) with 240 mcg of RPA563 on days 1, 15 and 29 (samples collected pre-dose) through study Day 42; n=10 animals per sex for all timepoints except Day 42 (n=2 per sex). Upper panel shows mean titer (units/mL) for male (●) and female (▪) rabbits, with dashed line indicating that only 5 animals were evaluated at Day 42. The bottom panel shows anti-PA titer for each individual animal in the study for each timpoint sampled. Dashed lined from Days 29-42 indicates a subset of animals (n=5 vs. n=10 for primary study).

### DETAILED DESCRIPTION

All publications, patents and patent applications cited herein, whether supra or infra, are hereby incorporated by reference in their entireties.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that, as used in this specification and the appended claims, the singular forms “a”, “an” and “the” include plural referents unless the content clearly dictates otherwise. Thus, for example, reference to “an agonist” includes a mixture of two or more such agonists, and the like.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, the terms “mcg” and “g” refer to micrograms.

### Vaccine Composition

Provided herein is an immunogenic composition comprising: (a) 5 mcg to 100 mcg B. anthracis rPA or mrPA protein and (b) liposome-embedded MPLA adjuvant, where the “r” in the name of the protein indicates that the protein is made recombinantly, i.e., using a recombinant system in a species that is not B. anthracis, e.g., in a protease deficient strain of P. fluorescens, as described below. In some embodiments, the protein may be expressed as fusion protein, purified and then cleaved from the fusion partner.

If the composition comprises rPA, then the composition may contain at least the C-terminal 139 amino acids of the carboxy terminus of the full length wild type PA polypeptide. The amino acid sequence of full length mature wild type rPA, i.e. lacking the 29 amino acid signal peptide, is shown below:

(SEQ ID NO: 1)
EVKQENRLLNESESSSQGLLGYYFSDLNFQAPMVVTSSTTGDLSI
PSSELENIPSENQYFQSAIWSGFIKVKKSDEYTFATSADNHVTMW
VDDQEVINKASNSNKIRLEKGRLYQIKIQYQRENPTEKGLDFKLY
WTDSQNKKEVISSDNLQLPELKQKSSNSRKKRSTSAGPTVPDRDN
DGIPDSLEVEGYTVDVKNKRTFLSPWISNIHEKKGLTKYKSSPEK
WSTASDPYSDFEKVTGRIDKNVSPEARHPLVAAYPIVHVDMENII
LSKNEDQSTQNTDSETRTISKNTSTSRTHTSEVHGNAEVHASFFD
IGGSVSAGFSNSNSSTVAIDHSLSLAGERTWAETMGLNTADTARL
NANIRYVNTGTAPIYNVLPTTSLVLGKNQTLATIKAKENQLSQIL
APNNYYPSKNLAPIALNAQDDFSSTPITMNYNQFLELEKTKQLRL
DTDQVYGNIATYNFENGRVRVDTGSNWSEVLPQIQETTARIIFNG
KDLNLVERRIAAVNPSDPLETTKPDMTLKEALKIAFGENEPNGNL
QYQGKDITEFDENFDQQTSQNIKNQLAELNATNIYTVLDKIKLNA
KMNILIRDKRFHYDRNNIAVGADESVVKEAHREVINSSTEGLLLN
IDKDIRKILSGYIVEIEDTEGLKEVINDRYDMLNISSLRQDGKTF
IDFKKYNDKLPLYISNPNYKVNVYAVTKENTIINPSENGDTSTNG
IKKILIFSKKGYEIG

The C-terminal 139 amino acids of PA contain the host cell receptor binding region (Little et al, Microbiology 1996 142: 707-715), have been shown to be essential for host cell intoxication (Varughese et al, Infect. Immun. 1999 67:1860-1865; Brossier et al Infect. Immun. 1999 67: 964-967), and contain the dominant protective epitopes of PA (Flick-Smith et al, Infect Immun. 2002 70: 1653-1656). In some embodiments, the rPA of the composition may have the amino acid sequence of SEQ ID NO:1.

If the composition comprises mrPA (or “mutant” rPA), then the mrPA may comprise mutations that abolish cleavage by furin or by chymotrypsin or by both chymotrypsin and furin. mrPA may contain the change SNSS167, SNKE167 or EGG167 (as described in Klimpel et al. Proc Natl Acad Sci 1992 89: 10277-81). mrPA may mrPA may be composed of the amino acid sequence of SEQ ID NO: 2, which is similar to SEQ ID NO: 1 except that it contains the following changes: SNKE167, ΔFF315 and E308D. The underlined sequence of RKKR above in SEQ ID NO: 1 is changed to SNKE as underlined in SEQ ID NO: 2 below. The amino acids FF, bold underlined above in SEQ ID NO: 1, are removed between the bold underlined S and D in SEQ ID NO: 2 below. The amino acid E, italicized and underlined in SEQ ID NO: 1 above, is changed to amino acid D, italicized and underlined in SEQ ID NO: 2 below.

(SEQ ID NO: 2)
EVKQENRLLNESESSSQGLLGYYFSDLNFQAPMVVTSSTTGDLSI
PSSELENIPSENQYFQSAIWSGFIKVKKSDEYTFATSADNHVTMW
VDDQEVINKASNSNKIRLEKGRLYQIKIQYQRENPTEKGLDFKLY
WTDSQNKKEVISSDNLQLPELKQKSSNSSNKESTSAGPTVPDRDN
DGIPDSLEVEGYTVDVKNKRTFLSPWISNIHEKKGLTKYKSSPEK
WSTASDPYSDFEKVTGRIDKNVSPEARHPLVAAYPIVHVDMENII
LSKNEDQSTQNTDSETRTISKNTSTSRTHTSEVHGNADVHASDIG
GSVSAGFSNSNSSTVAIDHSLSLAGERTWAETMGLNTADTARLNA
NIRYVNTGTAPIYNVLPTTSLVLGKNQTLATIKAKENQLSQILAP
NNYYPSKNLAPIALNAQDDFSSTPITMNYNQFLELEKTKQLRLDT
DQVYGNIATYNFENGRVRVDTGSNWSEVLPQIQETTARIIFNGKD
LNLVERRIAAVNPSDPLETTKPDMTLKEALKIAFGENEPNGNLQY
QGKDITEFDFNEDQQTSQNIKNQLAELNATNIYTVLDKIKLNAKM
NILIRDKRFHYDRNNIAVGADESVVKEAHREVINSSTEGLLLNID
KDIRKILSGYIVEIEDTEGLKEVINDRYDMLNISSLRQDGKTFID
FKKYNDKLPLYISNPNYKVNVYAVTKENTIINPSENGDTSTNGIK
KILIFSKKGYEIG

mrPA SNKE167, ΔFF315 and E308D is described in U.S. Pat. Nos. 7,261,900 and 8,044,189, which are incorporated by reference herein. In some embodiments, the composition may contain 5-15 mcg, 8-12 mcg, 9-11 mcg (e.g., 10 mcg), 45-55 mcg (e.g., 50 mcg), or 75-85 mcg (e.g., 80 mcg) of rPA or mrPA. In some embodiments, the composition may contain about 5 mcg, about 6 mcg, about 7 mcg, about 8 mcg, about 9 mcg, 10 mcg, about 11 mcg, about 12 mcg, about 13 mcg, about 14 mcg, about 15 mcg, about 20 mcg, about 25 mcg, about 30 mcg, about 40 mcg, about 45 mcg, about 46 mcg, about 47 mcg, about 48 mcg, about 49 mcg, about 50 mcg, about 51 mcg, about 52 mcg, about 53 mcg, about 54 mcg, about 55 mcg, about 56 mcg, about 57 mcg, about 58 mcg, about 59 mcg, about 60 mcg, about 65 mcg, about 70 mcg, about 75 mcg, about 76 mcg, about 77 mcg, about 78 mcg, about 79 mcg, about 80 mcg, about 81 mcg, about 82 mcg, about 83 mcg, about 84 mcg, about 85 mcg, or about 90 mcg or rPA or mrPA.

In some embodiments, the composition may comprise 5 mcg to 200 mcg (or 20 mcg to 200 mcg) liposome-embedded MPLA adjuvant, e.g., 5-10 mcg, 10-20 mcg, 20-30 mcg, 30-40 mcg, 40-60 mcg, 60-80 mcg, 80-100 mcg, 100-125 mcg, 125-150 mcg, or 150-200 mcg of liposome-embedded MPLA adjuvant, although the composition may contain more or less than this amount of liposome-embedded MPLA adjuvant in certain cases. In some embodiments, the composition may comprise 23 mcg to 27 mcg (e.g., 25 mcg), 45 to 55 mcg (e.g., 50 mcg) or 160 mcg to 180 mcg (e.g., 170 mcg) liposome-embedded MPLA adjuvant. In some embodiments, the composition may contain about 5 mcg, about 10 mcg, about 15 mcg, about 20 mcg, about 21 mcg, about 22 mcg, about 23 mcg, about 24 mcg, about 25 mcg, about 26 mcg, about 27 mcg, about 28 mcg, about 29 mcg, about 30 mcg, about 35 mcg, about 40 mcg, about 45 mcg, about 46 mcg, about 47 mcg, about 48 mcg, about 49 mcg, about 50 mcg, about 51 mcg, about 52 mcg, about 53 mcg, about 54 mcg, about 55 mcg, about 60 mcg, about 65 mcg, about 70 mcg, about 75 mcg, about 80 mcg, about 85 mcg, about 90 mcg, about 95 mcg, about 100 mcg, about 105 mcg, about 110 mcg, about 115 mcg, about 120 mcg, about 125 mcg, about 130 mcg, about 135 mcg, about 140 mcg, about 145 mcg, about 150 mcg, about 155 mcg, about 160 mcg, about 165 mcg, about 170 mcg, about 180 mcg, about 185 mcg, about 190 mcg, about 195 mcg or about 200 mcg liposome embedded MPLA adjuvant. MPLA is a toll-like receptor-4 agonist that is a non-pyrogenic form of lipopolysaccharide (Casella et al. Cell Mol. Life Sci. 2008 65: 3231-3240). MPLA can activate antigen-presenting cells for enhancing immune responses (Bohannon Shock 2013 40: 451-462). The MPLA used in the present system is a pure, synthetically made compound (as opposed to a mixture of lipids). Synthetic MPLA can be purchased from a variety of vendors, including Avanti Polar Lipids (Alabaster, AL) and has the following structure:

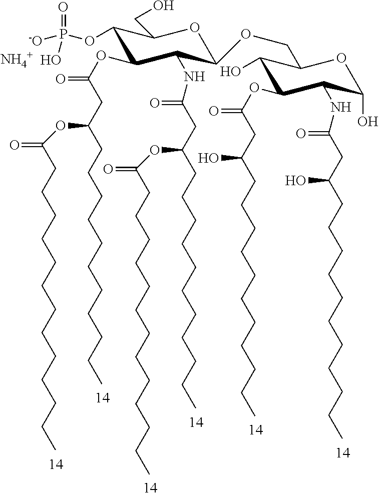

In some embodiments, the liposome-embedded MPLA adjuvant may be composed of dimyristoyl phosphatidylcholine (DMPC), dimyristoyl phosphatidylglycerol (DMPG) and cholesterol (at a 9:7:1 ratio, by weight).

The composition described above may be in liquid form and formulated for administration to a human subject. For example, the composition may be formulated in phosphate-buffered sucrose, pH 6.5. In these embodiments, the composition may be considered a “dose” of the vaccine, i.e., a measured quantity of a vaccine to be administered at one time. In these embodiments, a dose may have a volume of 5 ul, 10 ul, 25 ul, 50 ul 100, 150 ul 200 uL, 250 ul, 500 ul, 1 ml or 2 ml, for example.

A multi-dose vial is also provided. In these embodiments, the vial may contain a liquid formulation from which single doses of the vaccine can be drawn, e.g., by a syringe. A multi-dose vial may contain at least 5, at least 10, at least 20, at least 50 or at least 100 doses of the vaccine.

In these embodiments, the mrPA:MPLA or rPA:MPLA ratio (by weight) of the liquid composition may be about 10:1. In this embodiment, the composition may be formulated such that the volume of the liquid formulation administered to a patient (e.g., 200 ul, 250 ul, 500 ul, 1 ml or 2 ml) contains a single dose of 10 mcg mrPA or rPA and 5 mcg MPLA.

In these embodiments, the mrPA:MPLA or rPA:MPLA ratio (by weight) of the liquid composition may be about 10:10. In this embodiment, the composition may be formulated such that the volume of the liquid formulation administered to a patient (e.g., 200 ul, 250 ul, 500 ul, 1 ml or 2 ml) contains a single dose of 10 mcg mrPA or rPA and 10 mcg MPLA.

In these embodiments, the mrPA:MPLA or rPA:MPLA ratio (by weight) of the liquid composition may be about 10:25. In this embodiment, the composition may be formulated such that the volume of the liquid formulation administered to a patient (e.g., 200 ul, 250 ul, 500 ul, 1 ml or 2 ml) contains a single dose of 10 mcg mrPA or rPA and 25 mcg MPLA.

In other embodiments, the mrPA:MPLA or rPA:MPLA ratio (by weight) of the liquid composition may be about 10:50. In this embodiment, the composition may be formulated such that the volume of the liquid formulation administered to a patient (e.g., 200 ul, 250 ul, 500 ul, 1 ml or 2 ml) contains a single dose of 10 mcg mrPA or rPA and 50 mcg MLPA.

In other embodiments, the mrPA:MPLA or rPA:MPLA ratio (by weight) of the liquid composition may be about 10:170. In this embodiment, the composition may be formulated such that the volume of the liquid formulation administered to a patient (e.g., 200 ul, 250 ul, 500 ul, 1 ml or 2 ml) contains a single dose of 10 mcg mrPA or rPA and 170 mcg MLPA.

In other embodiments, the mrPA:MPLA or rPA:MPLA ratio (by weight) of the liquid composition may be about 50:25. In this embodiment, the composition may be formulated such that the volume of the liquid formulation administered to a patient (e.g., 200 ul, 250 ul, 500 ul, 1 ml or 2 ml) contains a single dose of 50 mcg mrPA or rPA and 25 mcg MLPA.

In other embodiments, the mrPA:MPLA or rPA:MPLA ratio (by weight) of the liquid composition may be about 50:50. In this embodiment, the composition may be formulated such that the volume of the liquid formulation administered to a patient (e.g., 200 ul, 250 ul, 500 ul, 1 ml or 2 ml) contains a single dose of 50 mcg mrPA or rPA and 50 mcg MLPA.

In other embodiments, the mrPA:MPLA or rPA:MPLA ratio (by weight) of the liquid composition may be about 50:170. In this embodiment, the composition may be formulated such that the volume of the liquid formulation administered to a patient (e.g., 200 ul, 250 ul, 500 ul, 1 ml or 2 ml) contains a single dose of 50 mcg mrPA or rPA and 170 mcg MLPA.

In other embodiments, the mrPA:MPLA or rPA:MPLA ratio (by weight) of the liquid composition may be about 80:25. In this embodiment, the composition may be formulated such that the volume of the liquid formulation administered to a patient (e.g., 200 ul, 250 ul, 500 ul, 1 ml or 2 ml) contains a single dose of 80 mcg mrPA or rPA and 25 mcg MLPA.

In other embodiments, the mrPA:MPLA or rPA:MPLA ratio (by weight) of the liquid composition may be about 80:50. In this embodiment, the composition may be formulated such that the volume of the liquid formulation administered to a patient (e.g., 200 ul, 250 ul, 500 ul, 1 ml or 2 ml) contains a single dose of 80 mcg mrPA or rPA and 50 mcg MLPA.

In other embodiments, the mrPA:MPLA or rPA:MPLA ratio (by weight) of the liquid composition may be about 80:170. In this embodiment, the composition may be formulated such that the volume of the liquid formulation administered to a patient (e.g., 200 ul, 250 ul, 500 ul, 1 ml or 2 ml) contains a single dose of 80 mcg mrPA or rPA and 170 mcg MLPA.

In some embodiments, the single dose may be formulated such that the composition of the liquid formulation contains about 10 mcg to 80 mcg rPA or mrPA and about 5 mcg to 170 mcg of MPLA. In these embodiment, the composition may be formulated such that the volume of the liquid formulation administered to a patient (e.g., 200 ul, 250 ul, 500 ul, 1 ml or 2 ml) contains a single dose of 15 mcg to 250 mcg mrPA or rPA and MPLA combined.

In some embodiments, the composition may be formulated such that the volume of the liquid formulation administered to a patient (e.g., 200 ul, 250 ul, 500 ul, 1 ml or 2 ml) contains a single dose of 15 mcg mrPA or rPA and MPLA combined.

In some embodiments, the composition may be formulated such that the volume of the liquid formulation administered to a patient (e.g., 200 ul, 250 ul, 500 ul, 1 ml or 2 ml) contains a single dose of 250 mcg mrPA or rPA and MPLA combined.

An immunogenic composition that comprises 60 mcg to 600 mcg B. anthracis mrPA (mutant rPA) protein and is free of adjuvant (i.e., a composition to which no adjuvant has been added) is also provided. This composition should be an injectable liquid. In some embodiments, the composition may contain 60 mcg to 600 mcg B. anthracis mrPA protein and an inert pharmaceutically acceptable excipient such as saline or phosphate-buffered sucrose. In some embodiments, the compostions may contain 60 mcg to 600 mcg B. anthracis mrPA protein and a buffer that contains 7.5-8.5 mM sodium phosphate, 1.0-2.0 mM potassium phosphate, 2.5-3.0 mM KCl, and 250-300 mM sucrose at a pH in the range of 6.0-7.0. For example the buffer may contain 8.1 mM sodium phosphate, 1.5 mM potassium phosphate, 2.7 mM KCl, and 270 mM sucrose at pH6.5. In some embodiments, the composition may comprise 70-90 mcg (e.g., 80 mcg), 140-180 mcg (e.g., 160 mcg) or 220-260 mcg (e.g., 240 mcg) mrPA protein. In some embodiments the composition may comprise 70 mcg, 75 mcg, 80 mcg, 85 mcg, 90 mcg, 95 mcg, 100 mcg, 105 mcg, 110 mcg, 115 mcg, 120 mcg, 125 mcg, 130 mcg, 135 mcg, 140 mcg, 145 mcg, 150 mcg, 155 mcg, 160 mcg, 165 mcg, 170 mcg, 175 mcg, 180 mcg, 185 mcg, 190 mcg, 195 mcg, 200 mcg, 205 mcg, 210 mcg, 215 mcg, 220 mcg, 225 mcg, 230 mcg, 235 mcg, 240 mcg, 245 mcg, 250 mcg, 255 mcg, or 260 mcg mrPA protein. In any embodiment, the mrPA protein may comprise the amino acid sequence of SEQ ID NO. 2.

### Methods of Use

Provided herein is a method for inducing an immune response to B. anthracis. This method may comprise administering a first dose of the immunogenic composition to a subject, e.g., a human subject. In these embodiments, the immune response may be a protective immune response, thereby protecting the subject from disease resulting from exposure to B. anthracis spores. Administration of the immunogenic composition to the subject induces the production of toxin neutralizing antibodies (TNA), i.e., antibodies that bind to B. anthracis PA and prevent it from binding to the host cell receptor. The level of TNA produced may correlate with the TNA NF₅₀ (50% neutralization factor) threshold of 0.56 observed in rabbits and/or 0.29 in non-human primates.

In some embodiments, the present immunogenic composition may be indicated is a vaccine for the active immunization for the prevention of disease caused by Bacillus anthracis in persons 18 through 65 years of age, particularly for pre-exposure prophylaxis of disease in persons whose occupation or other activities place them at high risk of exposure. The present immunogenic composition may also be indicated for post-exposure prophylaxis of disease following suspected or confirmed Bacillus anthracis exposure, particularly when administered in conjunction with recommended antibacterial drugs.

Although a certain degree of protection may be obtained from a single dose of the composition, one or more booster shots may be necessary to obtain a sustained protection (i.e., a protection that may last for years). In these embodiments, the method may comprise administering a second dose of the immunogenic composition to the subject, wherein the first and second doses are administered about 2-5 weeks apart, e.g., 13-15 days apart, or 26-30 days apart. Further doses (e.g., a third dose and possibly a fourth dose) may be administered at the same time interval as the first and second dose in some cases. In some embodiments, a sustained protection against disease caused by B. anthracis may be obtained by two doses. In some embodiments, a sustained protection against disease caused by B. anthracis may be obtained by one dose.

Each dose of the immunogenic composition may be administered by intramuscular injection (e.g., into the deltoid muscle of the arm, the vastus lateralis muscle of the thigh, the ventrogluteal muscle of the hip or the dorsogluteal muscles of the buttock), although other administration routes may be used in certain circumstances. In some embodiments the immunogenic composition may be administered by intradermal or subcutaneous injection. In some embodiments the immunogenic composition may be administered by a transdermal patch.

In some embodiments, the first dose may be administered to the subject after the subject is exposed to a source of B. anthracis spores, e.g., up to 24 or 48 hrs after the subject has come into contact with a powder, spray, food, water or the contents of a bioterrorism threat that contains B. anthracis spores or is thought to contain B. anthracis spores, or come in contact with an infected individual. In other embodiments, the first dose may be administered to the subject before the subject is exposed to B. anthracis spores, e.g., in advance of combat against an enemy force that is known to have weaponized B. anthracis.

In some embodiments, the method may further comprise administering antibiotics (e.g., ciprofloxacin, doxycycline and/or levofloxacin) to the subject, after the first dose. In some embodiments, a course of the antibiotics may be administered to the subject, starting, e.g., 30-40 days after the first dose.

### Examples

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Materials and Methods

### Study Design

Study PF563-101 (ClinicalTrials.gov identifier NCT02655549) was a double-blind, placebo-controlled, dose-escalation trial in 54 subjects. Eligible study participants were healthy male and female subjects who were 18-55 years-old and able to understand and sign an Institutional Review Board-approved informed consent form. Subjects with a history of anthrax disease or having received any licensed or investigational anthrax vaccine or treatment (e.g., monoclonal antibodies, anthrax immune globulin) were ineligible for study participation.

Three dose levels of Px563L (10, 50, and 80 mcg mrPA) were investigated. Each dose level of Px563L was formulated with 0.17 mg of MPLA embedded in a liposome. RPA563 (mrPA only) and placebo (saline) were used as controls in the study. An MPLA-liposome only control group was not included, since AE contributions from the formulation can be inferred by comparing the profiles of Px563L vs RPA563. For each dose level, 18 subjects were randomized in an 8:8:2 ratio to Px563L, RPA563, or placebo. Each subject received an IM injection in alternating deltoid muscles on Day 0 and Day 28.

The Investigator, Sponsor's Medical Monitor, site personnel conducting safety assessment, and all subjects were blinded to treatment assignment. Vaccine preparation and administration were performed by unblinded study personnel, and study participants were masked during vaccination.

Following the Day 7 visit for all subjects at a dose level, a review of blinded clinical and safety laboratory data was conducted by the Safety Review Committee (SRC), which included the Investigator and Sponsor's Medical Monitor. The SRC permitted dose escalation to the next planned level if no clinically significant findings were observed. A safety and immunogenicity analysis was conducted after all subjects completed the Day 70 visit, with all subjects followed up to Day 393 for long-term safety assessment.

This study was conducted at a single study center in the United States and in accordance with standards of Good Clinical Practice, as defined by the International Council for Harmonisation (ICH) and all applicable federal and local regulations.

### Investigational Products

The mrPA protein was manufactured under current Good Manufacturing Practice conditions from a recombinant P. fluorescens production strain in a characterized mineral salts and glycerol growth medium free of animal-derived products, followed by cell collection, cell lysis and clarification, protein purification, ultrafiltration, and formulation in phosphate-buffered sucrose.

Px563L drug product was manufactured as a sterile liquid consisting of 400 mcg/mL of mrPA in phosphate-buffered sucrose, pH 6.5, and 0.85 mg/mL of adjuvant (synthetic MPLA embedded in a liposome composed of DMPC, cholesterol, and DMPG in a 9:7:1 ratio).

RPA563 drug product was manufactured as a sterile liquid consisting of 400 mcg/mL of mrPA in phosphate-buffered sucrose, pH 6.5.

Placebo injection was a sterile saline for injection (0.9% sodium chloride).

Px563L and RPA563 were supplied in injection vials. The injection volume for all 3 dose levels was 0.2 mL. Px563L and RPA563 were diluted with MPLA-liposome or saline, respectively, to deliver the required amount of mrPA for the 10 and 50 mcg dose levels.

### Safety Assessment

On vaccination days, subjects were observed for AEs in the study center for approximately 30 minutes following vaccination. Daily temperatures and reports of local and systemic AEs were recorded by the subject in a diary for 7 days after each vaccination. The diary was returned to the study center on Day 7 and Day 35 and reviewed for AEs and changes in concomitant medication use. Subjects were provided with a thermometer and ruler, as well as instructions, to assess daily body temperature and measure any erythema or induration at the injection site. Serum chemistry, hematology, urinalysis and electrocardiogram assessments were performed at clinic visits through Day 70.

The severity of all AEs was assessed as Grade 1 (mild), 2 (moderate), 3 (severe), or 4 (potentially life-threatening), as defined in the toxicity grading guidance for vaccine trials by the U.S. Food and Drug Administration (FDA, 2007). AEs were coded using the Medical Dictionary for Regulatory Activities, version 18.1.

### Immunogenicity Assessment

Serum samples were collected on Days 0 (before vaccination), 7, 14, 28, 35, 42, 56 and 70. Samples were tested using a validated, high-throughput, cell-based bioassay (Battelle Memorial Institute, Columbus, OH, USA) to measure the neutralization of anthrax lethal toxin by antibodies generated in response to vaccination (Li, 2008; Omland, 2008). The TNA NF₅₀ value was determined as the ratio of effective dilution 50% (ED₅₀) of a particular test sample to the ED₅₀ of a human reference serum AVR801 (Semenova, 2004) from subjects vaccinated with the currently licensed product (BioThrax).

### Statistical Methods

All subjects who received any amount of investigational product (Px563L, RPA563, or placebo) were included in the analyses. Statistical summaries were descriptive in nature (e.g., means, standard deviations, and percentiles). All subjects were grouped according to treatment received, and missing data were not imputed. Subject disposition was tabulated for each study treatment, and all placebo-treated subjects were combined into one placebo group. The pre-specified analysis populations were (1) safety population, consisting of all subjects who received at least 1 dose of investigational product, and (2) immunogenicity population, consisting of all subjects with data up to and including Day 70. All 54 subjects were included in both analysis populations.

Immunogenicity data were summarized using descriptive statistics. Geometric mean (GM) and 95% two-sided confidence interval (CI) for GM were computed. Data below the limit of quantification were replaced by half of the lower limit of quantification for summary statistics. The 95% CI of the proportion of human subjects achieving a TNA NF₅₀ threshold was determined by the Jeffreys method. Because all immunogenicity data were collected within protocol-allowed time windows, no samples were excluded from summary statistical analysis.

### Results

### Subject Demographics and Disposition

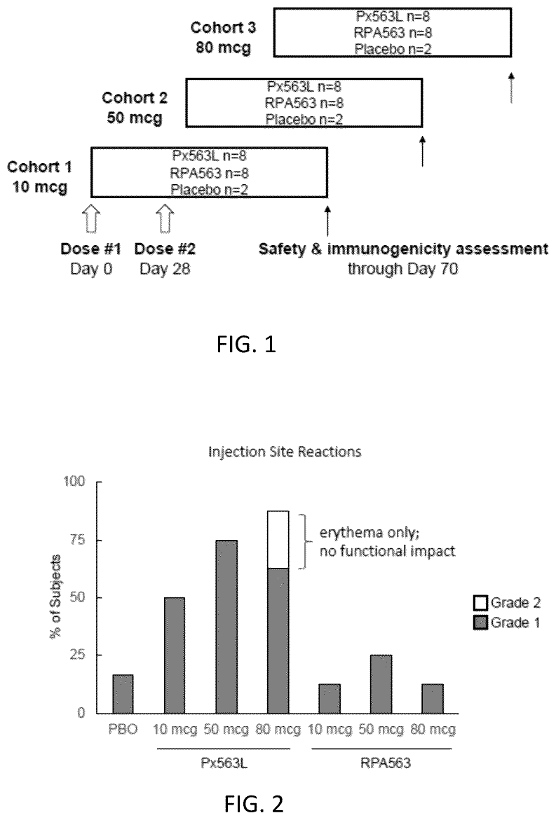

A total of 54 subjects were enrolled: 18 subjects to Cohort 1 (10 mcg), 18 subjects to Cohort 2 (50 mcg), and 18 subjects to Cohort 3 (80 mcg). Within each cohort, 8 subjects received Px563L, 8 subjects received RPA563, and 2 subjects received placebo (FIG. 1). There were 30 (55.6%) female subjects and 24 (44.4%) male subjects. Most of the subjects were white (36, or 66.7%), and the overall mean age of the subjects was 35.5 years (range of 19 to 54). There were no SAEs, AEs of potential autoimmune etiology, or AEs leading to study discontinuation through Day 70. All subjects completed the Day 70 safety assessment and immunogenicity blood draw.

### Safety and Tolerability

Overall, 36 (75%) subjects on active treatment (Px563L or RPA563) and one (16.7%) subject on placebo reported at least one AE through Day 70.

For Px563L, the number (%) of subjects reporting at least one treatment-related AE were 5 (62.5%) for 10 mcg, 7 (87.5%) for 50 mcg, and 8 (100%) for 80 mcg. For RPA563, the number (%) of subjects reporting at least one treatment-related AE were 2 (25%) for 10 mcg, 2 (25%) for 50 mcg, and 1 (12.5%) for 80 mcg.

The most common treatment-related AEs were injection site reactions, reported by a total of 21 (38.9%) subjects. Only Grade 1 injection site reactions were observed with Px563L at 10 and 50 mcg (FIG. 2). Although a Grade 2 injection site reaction (due to the size of erythema) was reported by two subjects receiving 80 mcg of Px563L, the findings were transient and not associated with limitations in arm motion or other functional impairment.

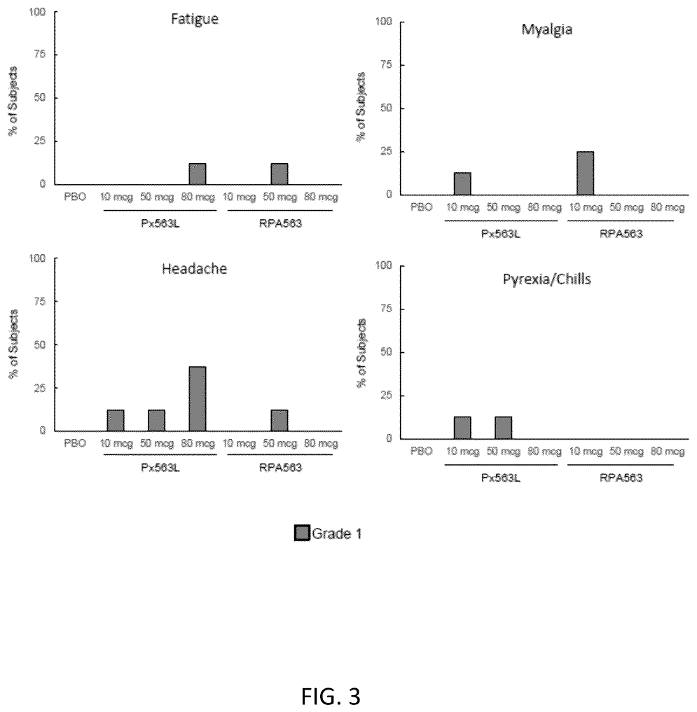

Only Grade 1 systemic reactogenicity AEs (fatigue, headache, myalgia, and pyrexia/chills) were observed with Px563L at any dose level (FIG. 3). Other systemic AEs reported through Day 70 included upper respiratory tract infection, diarrhea, dizziness, and hyperhidrosis, each of which was reported by <10% of all study subjects. There were no imbalances in the distribution of these systemic AEs across different dose levels of Px563L.

There were no clinically significant changes in serum chemistry, hematology, urinalysis or electrocardiogram assessments in any of the treatment groups.

### Immunogenicity

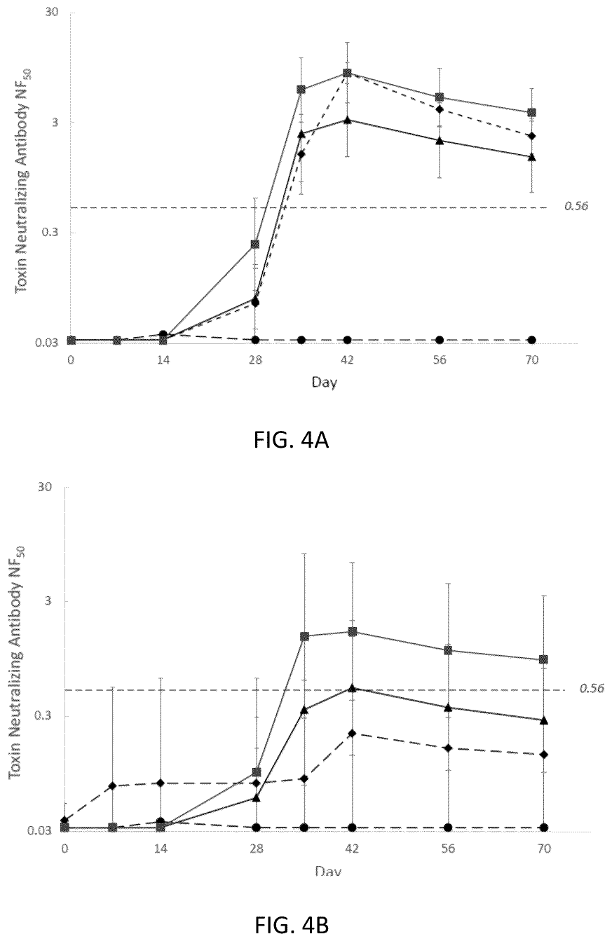

For the primary immunogenicity endpoint of TNA NF₅₀, titers started to increase significantly after the second dose of Px563L at all dose levels (FIG. 4). Beginning at Day 35, the GM and lower bound of the 95% CI for all doses of Px563L exceeded 0.56—a level correlated with significant survival in animal models of anthrax exposure (Ionin, 2013) and established as a relevant clinical and regulatory benchmark by BioThrax (Hopkins, 2014; Hopkins, 2016). The GM and lower bound of the 95% CI for all doses of Px563L remained above 0.56 through Day 70.

RPA563 exhibited a less robust immunogenicity response relative to Px563L. None of the RPA563 doses achieved a TNA NF₅₀ profile in which both the GM and lower bound of the 95% CI exceeded 0.56 from Day 35 through Day 70.

Because the approval of BioThrax for post-exposure prophylaxis was based on the percentage of subjects with TNA NF₅₀ values≥0.56 (Hopkins, 2014; BioThrax package insert, 2015), calculated the effects of Px563L were calculated at Day 70 (Table 1). Px563L in a two-dose regimen achieved a TNA NF₅₀ titer≥0.56 in 87.5 to 100% of subjects, and the lower bound of the 95% CI of the proportion of subjects achieving the TNA NF₅₀ threshold was 55 to 74%. For comparison, a three-dose regimen of BioThrax has been shown to achieve a TNA NF₅₀ titer≥0.56 in 57.9% of subjects, and the lower bound of the 95% CI was 50.4% (Hopkins, 2014).

TABLE 1

| | % subjects with | |
|---|---|---|
| Product | TNA | Lower limit of |
| and dose | NF₅₀ ≥0.56 | 95% CI |
| Px563L 10 mcg | 100.0% | 74% |
| Px563L 50 mcg | 87.5% | 55% |
| Px563L 80 mcg | 100.0% | 74% |
| BioThrax | 57.9% | 50.4% |
| 3 doses (SC) | | |

### Preclinical Study in Rabbits

A preclinical study was conducted in NZW rabbits to evaluate the efficacy of Px563L at different dose concentrations and vaccination schedules against inhalation anthrax following aerosol challenge with B. anthracis Ames spores. Sera were collected weekly, and immunological responses were monitored via the B. anthracis lethal toxin neutralization assay (TNA). Nine groups of 12 (6 male and 6 female per group, total n=108) NZW rabbits were immunized on Day 0. Most groups received a boost on either Day 14 or 28. Rabbits were challenged on Day 70 with a lethal dose of B. anthracis Ames spores (group means of 149 to 238 LD₅₀ equivalents).

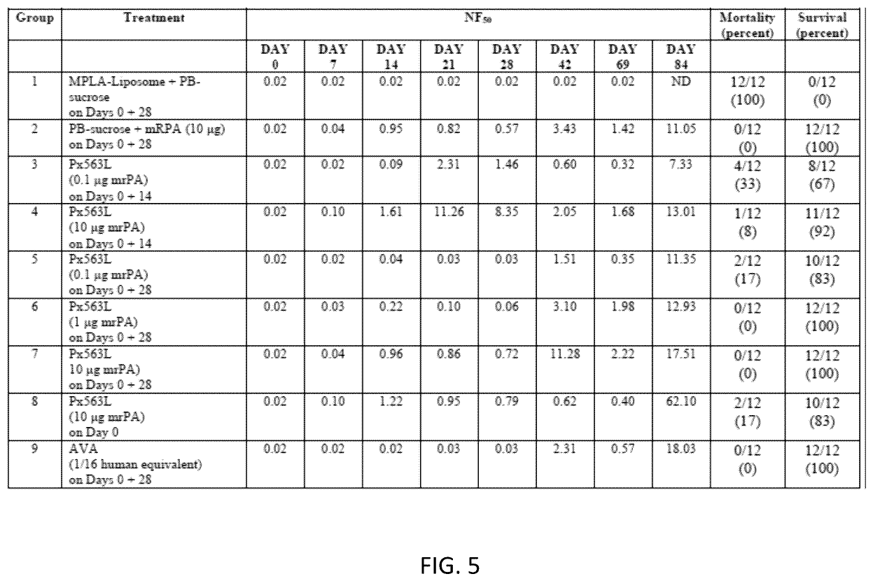

As shown in FIG. 5, the two groups vaccinated with Px563L containing 1.0 or 10 mcg mrPA on Days 0 and 28 (Group #6 and #7) were fully protected from lethal anthrax challenge. Vaccination with 10 mcg mrPA formulated with PB-sucrose vehicle (RPA563) or anthrax vaccine adsorbed (AVA) at Days 0 and 28 (Group #2 and #9) also provided full protection from lethal challenge with B. anthracis spores on Day 70. All vehicle/MPLA-liposome control rabbits (Group #1) succumbed to inhalational anthrax by Day 76. All other Px563L regimens (containing mrPA<1 μg or alternative dosing schedules) provided partial, although still significant, protection against lethal anthrax challenge.

As shown in FIG. 5, there was a general dose-response, with survival correlating with the concentration of mrPA contained in the Px563L formulation and TNA [i.e. higher concentrations of mrPA yielded higher 50% effective dilution (ED₅₀) values]. Individual NF₅₀ values from Day 42 and 69, along with survival data, were log transformed and used to perform a linear regression analysis. Pre-exposure NF₅₀ values of 0.38 and 0.22 for Days 42 and 69, respectively, were correlated with a 70% probability of survival P<0.0001).

The above-described Phase 1 study was designed to assess the safety and immunogenicity of Px563L, an adjuvanted recombinant anthrax vaccine candidate, in a two-dose regimen in healthy adult subjects. It was shown that that Px563L at all dose levels was safe and generated a robust immunogenicity response, as measured by TNA NF₅₀ titers, starting at Day 35 (one week after the second dose) and lasting through at least Day 70.

The most common treatment-related AEs associated with Px563L were injection site reactions, which were mostly mild. Only Grade 1 (mild) injection site reactions were observed with Px563L at 10 and 50 mcg dose levels, and the two Grade 2 (moderate) injection site erythemas associated with Px563L at 80 mcg did not result in arm motion limitation or other functional impairment. For comparison, after subcutaneous (SC) administration of BioThrax, up to 13.8% and 2.0% of subjects reported moderate and severe arm motion limitation, respectively (Hopkins, 2014). As another basis of comparison, up to 15% of subjects reported moderate arm motion limitation after IM administration of NuThrax™ (AV7909), which is a BioThrax-based vaccine candidate being developed for post-exposure prophylaxis (Hopkins, 2016). In addition, in a study including adults≥66 years of age, up to 45% of subjects reported injection site movement impairment after two doses of NuThrax (Wolfe, 2020).

Px563L was associated with minimal systemic AEs, and only Grade 1 (mild) systemic reactogenicity AEs (fatigue, headache, myalgia, and pyrexia/chills) were observed in 12.5% of subjects at 10 mcg, 12.5% of subjects at 50 mcg, and 37.5% of subjects at 80 mcg. For comparison, approximately 22% to 56% of subjects reported a systemic reactogenicity AE after a SC administration of BioThrax (Hopkins, 2014), including up to 13.8% and 1.0% of subjects who reported moderate and severe episodes, respectively. As another basis of comparison, up to 60% of subjects who received IM administration of NuThrax reported a systemic reactogenicity AE, including up to 10% who reported moderate severity (Hopkins, 2016).

Both the local and systemic AE findings associated with Px563L suggest a favorable safety and tolerability profile relative to BioThrax-based anthrax vaccine products. The relative lack of systemic and local reactogenicity findings with Px563L may be due to its controlled recombinant production process. In contrast, BioThrax is manufactured from filtered B. anthracis culture supernatants and contains small amounts of other bacterial components that may contribute to its reactogenicity (Jollenbeck, 2002).

A two-dose regimen of Px563L at all dose levels achieved a TNA NF₅₀ profile that exceeded the 0.56 threshold starting at Day 35 and lasting through Day 70. A TNA NF₅₀ titer of 0.56 has been shown to correspond to 70% and 88% probability of survival after anthrax exposure in rabbits and non-human primates, respectively (Ionin, 2013). These results show that a two-dose regimen of Px563L can achieve protective immunity seven (7) days after the second vaccination, and that the protective immunity can be sustained for at least another 35 days. The magnitude and duration of titer increase with Px563L appear at least as comparable to, and possibly superior to, a three-dose BioThrax regimen (Hopkins, 2014; Hopkins, 2016).

Results from an initial study indicate that a 70% survival level in rabbits is correlated with TNA NF₅₀ titer less than 0.56, indicating that this formulation may provide a higher level of protection than BioThrax.

Currently, the standard of medical practice for anthrax post-exposure prophylaxis is a three-dose SC regimen of BioThrax vaccine, administered at 0, 2, and 4 weeks (BioThrax package insert, 2015). While protective immunity is being generated, a concomitant antimicrobial regimen (e.g., ciprofloxacin, doxycycline) is prescribed for 42 days after first dose (Bower, 2019), so that any dormant anthrax spores that become activated during the time period can be eliminated. Px563L has the potential to shorten the duration of antibiotics from 42 to 35 days. Given that fewer than 50% of exposed individuals were compliant with post-exposure prophylactic measures during the 2001 anthrax attacks in the United States (Shepard, 2002), the improved safety, enhanced immunogenicity, and dose-sparing attributes of Px563L will likely increase subject compliance. Additionally, the decreased number of required vaccine and antimicrobial doses in a Px563L-based regimen should facilitate the efficiency and cost-effectiveness of local or national procurement efforts.

### Phase 1b/2 Trial Design

Phase 2 clinical trials will evaluate the efficacy of 10 mcg mPA protein combined with varying levels of liposomal MPLA (25-170 mcg), with a second dose at either 14 or 28 days.

The study design is shown in the table below.

| Attribute | Description |
|---|---|
| Study Title | Randomized, placebo controlled, double-blind, two-part, phase |
| | 1b/2 clinical study to assess the safety and immunogenicity of |
| | varying doses and regimens of Px563L and RPAS63 |
| Population | 365 adults from 18 to 65 years of age who are in stable health |
| | will participate in the study |
| Study Design | Dose/Regimen Selection (Part A): approximately 190 subjects |
| | will be enrolled into 6 active (n = 20) and 1 placebo (n = 10) |
| | cohorts |
| | Confirmation (Part B): approximately 175 subjects will be |
| | enrolled into 4 treatment groups (n = 35) |
| Design | This design affords an opportunity to combine results from Part |
| Rationale | A and Part B for a given dose/regimen, to guide accurate dose |
| | and regimen selection |
| Study Duration | Subjects are expected to be enrolled in the study for 14 months |
| Safety | Treatment emergent adverse events and serious adverse events |
| Outcomes | (Days: 0-393) |
| | Vital signs, ECGs, and clinical laboratory abnormalities (Days: |
| | 0-393) |
| | Local and systemic reactogenicity (Days: 0, 14, & 28) |
| Immunogenicity | TNA NF₅₀ antibody levels assessed at Days: 0, 7, 14, 21, 28, 35, |
| Outcomes | 42, , 49, 63, 84, 180, & 393 |
| | Seroconversion (Anti-PA IgG) assessed at Days: 0, 7, 14, 21, 28, |
| | 35, 42, 49, 63, 84, 180, & 393 |

The dose and regimen selection*(Pant A) of this study is shown in the table below.

| | | | Dose | | |
|---|---|---|---|---|---|
| | | Dose | (mcg | | Cohort size |
| Cohort # | Product | (mcg mrPA) | MPLA) | days | (n) |
| 1^{¥} | Px563L | 10 | 170 | 0, 28 | 20 |
| 2 | Px563L | 10 | 170 | 0, 14 | 20 |
| 3 | Px563L | 10 | 50 | 0, 28 | 20 |
| 4 | Px563L | 10 | 50 | 0, 14 | 20 |
| 5 | Px563L | 10 | 25 | 0, 28 | 20 |
| 6 | Px563L | 10 | 25 | 0, 14 | 20 |
| 7 | Placebo | n/a | n/a | 0, 28 | 10 |
| 8 | RPA563 | 80 | 0 | 0, 28 | 20 |
| | | | | (saline | |
| | | | | on Day | |
| | | | | 14) | |
| 9 | RPA563 | 160 | 0 | 0, 14 | 20 |
| | | | | (saline | |
| | | | | on say | |
| | | | | 28) | |
| 10 | RPA563 | 160 | 0 | 0, 28 | 20 |
| | | | | (saline | |
| | | | | on Day | |
| | | | | 14) | |

*Part B dose/regimen selection will be based on Part A safety (Day 0-60) and NF₅₀ data at Day 64
^{¥}This Dose/Regimen is selected from the Phase 1a clinical study

### Toxicology Studies

In the study described above (Preclinical Study in Rabbits), it was found that a 10 mcg dose of RPA563 at days 0 and 28 was found to be protective in New Zealand white rabbits (see FIG. 5, group 2). Follow up studies on RPA563 (i.e., mrPA in phosphate-buffered sucrose, pH 6.5, without adjuvant) were performed using the same animal model.

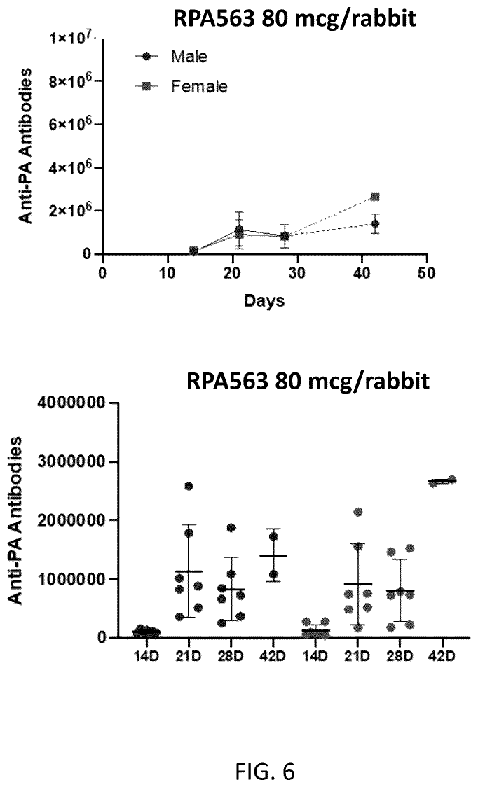

RPA563 containing 80 mcg mRPA was administered intramuscularly to rabbits on days 1, 14 and 28. In this study, n=7 animals per sex per group for all timepoints except 42 days (n=2 per sex). AntiPA antibodies were measured. Administration of RPA563 at a dose of 80 mcg induces an immune response in these animals. See FIG. 6.

Toxicology studies were performed with RPA563 (240 mcg mrPA) to investigate the safety of dosing at levels up to 160 mcg in the Phase 1b/2 clinical trial. Microscopic examination of Iliac lymph node and spleen noted physiologic response consistent with production of antibodies in response to mrPA administration This dose is well tolerated with occasional skin irritations noted in both vehicle and drug treated animals. The 240 mcg mrPA dose was determined to be a “no-observed-adverse-effects-level’ (NOAEL).

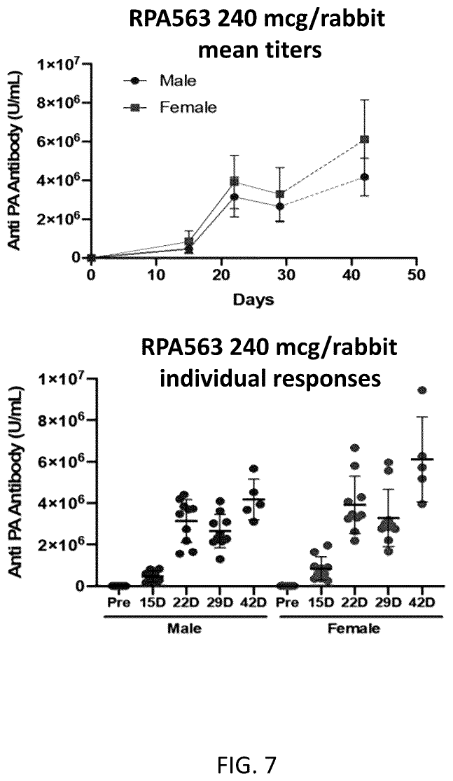

In this study, all animals demonstrated significant response to immunization with RPA563 at 240 mcg intramuscular dose on days 1, 15 and 29 vs a PBS control (n=10 per sex per group). All animals dosed with 240 mcg mrPA demonstrated an anti-PA antibody response (no non-responders). These data are shown in FIG. 7. In FIG. 7, the dashed lined from D29-42 indicates a subset of animals (n=5 vs. n=10 for primary study). Higher levels of anti-PA antibodies were observed with 240 mcg dose compared to 80 mcg dose in the previous toxicology/immunogenicity study.

### REFERENCES

BioThrax (Anthrax Vaccine Adsorbed) [package insert]. Lansing, MI, USA: Emergent BioSolutions, 2015.
Bower W A, Schiffer J, Atmar R L, Keitel W A, Friedlander A M, Liu L, Yu Y, Stephens D S, Quinn C P, Hendricks K; ACIP Anthrax Vaccine Work Group. Use of Anthrax Vaccine in the United States: Recommendations of the Advisory Committee on Immunization Practices, 2019. MMWR Recomm Rep. 2019 Dec. 13; 68(4):1-14. doi: 10.15585/mmwr.rr6804al. PMID: 31834290; PMCID: PMC6918956.
Hopkins R J, Howard C, Hunter-Stitt E, Kaptur P E, Pleune B, Muse D, Sheldon E, Davis M, Strout C, Vert-Wong K. Phase 3 trial evaluating the immunogenicity and safety of a three-dose BioThrax® regimen for post-exposure prophylaxis in healthy adults. Vaccine. 2014 Apr. 17; 32(19):2217-24. doi: 10.1016/j.vaccine.2014.01.073. Epub 2014 Mar. 6. PMID: 24613523.
Hopkins R J, Kalsi G, Montalvo-Lugo V M, Sharma M, Wu Y, Muse D D, Sheldon E A, Hampel F C, Lemiale L. Randomized, double-blind, active-controlled study evaluating the safety and immunogenicity of three vaccination schedules and two dose levels of AV7909 vaccine for anthrax post-exposure prophylaxis in healthy adults. Vaccine. 2016 Apr. 19; 34(18):2096-105. doi: 10.1016/j.vaccine.2016.03.006. Epub 2016 Mar. 12. PMID: 26979136; PMCID: PMC4839983.
Ionin B, Hopkins R J, Pleune B, Sivko G S, Reid F M, Clement K H, Rudge T L Jr, Stark G V, Imes A, Sari S, Guina T, Howard C, Smith J, Swoboda M L, Vert-Wong E, Johnson V, Nabors G S, Skiadopoulos M H. Evaluation of immunogenicity and efficacy of anthrax vaccine adsorbed for postexposure prophylaxis. Clin Vaccine Immunol. 2013 July;20(7):1016-26. doi: 10.1128/CVI.00099-13. Epub 2013 May 8. PMID: 23658392; PMCID: PMC3697458.
Institute of Medicine (US) Committee to Assess the Safety and Efficacy of the Anthrax Vaccine. The Anthrax Vaccine: Is It Safe? Does It Work? Joellenbeck L M, Zwanziger L L, Durch J S, Strom B L, editors. Washington (DC): National Academies Press (US); 2002. PMID: 25057597.
Li H, Soroka S D, Taylor T H Jr, Stamey K L, Stinson K W, Freeman A E, Abramson D R, Desai R, Cronin L X, Oxford J W, Caba J, Pleatman C, Pathak S, Schmidt D S, Semenova V A, Martin S K, Wilkins P P, Quinn C P. Standardized, mathematical model-based and validated in vitro analysis of anthrax lethal toxin neutralization. J Immunol Methods. 2008 Apr. 20; 333(1-2):89-106. doi: 10.1016/j.jim.2008.01.007. Epub 2008 Feb. 8. PMID: 18304568.
Omland K S, Brys A, Lansky D, Clement K, Lynn F; Participating Laboratories. Interlaboratory comparison of results of an anthrax lethal toxin neutralization assay for assessment of functional antibodies in multiple species. Clin Vaccine Immunol. 2008 June;15(6):946-53. doi: 10.1128/CVI.00003-08. Epub 2008 Apr. 16. PMID: 18417668; PMCID: PMC2446612.
Peachman K K, Li Q, Matyas G R, Shivachandra S B, Lovchik J, Lyons R C, Alving C R, Rao V B, Rao M. Anthrax vaccine antigen-adjuvant formulations completely protect New Zealand white rabbits against challenge with Bacillus anthracis Ames strain spores. Clin Vaccine Immunol. 2012 January;19(1):11-6. doi: 10.1128/CVI.05376-11. Epub 2011 Nov. 16. PMID: 22089245; PMCID: PMC3255956.
Ramirez D M, Leppla S H, Schneerson R, Shiloach J. Production, recovery and immunogenicity of the protective antigen from a recombinant strain of Bacillus anthracis. J Ind Microbiol Biotechnol. 2002 April; 28(4):232-8. doi: 10.1038/sj/jim/7000239. PMID: 11986925.
Rao M, Peachman K K, Li Q, Matyas G R, Shivachandra S B, Borschel R, Morthole V I, Fernandez-Prada C, Alving C R, Rao V B. Highly effective generic adjuvant systems for orphan or poverty-related vaccines. Vaccine. 2011 Jan. 29; 29(5):873-7. doi: 10.1016/j.vaccine.2010.11.049. Epub 2010 Nov. 27. PMID: 21115053; PMCID: PMC4190060.
Reed M D, Wilder J A, Mega W M, Hutt J A, Kuehl P J, Valderas M W, Chew L L, Liang B C, Squires C H. Immunization with a Recombinant, Pseudomonas fluorescens-Expressed, Mutant Form of Bacillus anthracis-Derived Protective Antigen Protects Rabbits from Anthrax Infection. PLoS One. 2015 Jul. 24; 10(7):e0130952. doi: 10.1371/journal.pone.0130952. PMID: 26207820; PMCID: PMC4514824.
Retallack D M, Jin H, Chew L. Reliable protein production in a Pseudomonas fluorescens expression system. Protein Expr Purif. 2012 February;81(2):157-65. doi: 10.1016/j.pep.2011.09.010. Epub 2011 Sep. 24. Erratum in: Protein Expr Purif 2012 Oct;85(2):251. PMID: 21968453.
Semenova V A, Steward-Clark E, Stamey K L, Taylor T H Jr, Schmidt D S, Martin S K, Marano N, Quinn C P. Mass value assignment of total and subclass immunoglobulin G in a human standard anthrax reference serum. Clin Diagn Lab Immunol. 2004 September;11(5):919-23. doi: 10.1128/CDLI.11.5.919-923.2004. PMID: 15358653; PMCID: PMC515271.
Shepard C W, Soriano-Gabarro M, Zell E R, Hayslett J, Lukacs S, Goldstein S, Factor S, Jones J, Ridzon R, Williams I, Rosenstein N; CDC Adverse Events Working Group. Antimicrobial postexposure prophylaxis for anthrax: adverse events and adherence. Emerg Infect Dis. 2002 October;8(10):1124-32. doi: 10.3201/eid0810.020349. PMID: 12396927; PMCID: PMC2730317.
Singh Y, Chaudhary V K, Leppla S H. A deleted variant of Bacillus anthracis protective antigen is non-toxic and blocks anthrax toxin action in vivo. J Biol Chem. 1989 Nov. 15; 264(32):19103-7. PMID: 2509473.
Singh Y, Klimpel K R, Arora N, Sharma M, Leppla S H. The chymotrypsin-sensitive site, FFD315, in anthrax toxin protective antigen is required for translocation of lethal factor. J Biol Chem. 1994 Nov. 18; 269(46):29039-46. PMID: 7961869.
U.S. Food and Drug Administration guidance for industry. Toxicity grading scale for healthy adult and adolescent volunteers enrolled in preventive vaccine clinical trials, September 2007.
Wagner L, Verma A, Meade B D, Reiter K, Narum D L, Brady R A, Little S F, Burns D L. Structural and immunological analysis of anthrax recombinant protective antigen adsorbed to aluminum hydroxide adjuvant. Clin Vaccine Immunol. 2012 Sep;19(9):1465-73. doi: 10.1128/CVI.00174-12. Epub 2012 Jul. 18. PMID: 22815152; PMCID: PMC3428410.
Wolfe D N, Espeland E M, Gao Y, Lu D, Blatner G, Amass K, Horwith G, Tong X M, Hopkins R, David G L, Jepson B M, King J C Jr. Evaluation of BioThrax® and AV7909 anthrax vaccines in adults 66 years of age or older. Vaccine. 2020 Nov. 25; 38(50):7970-7976. doi: 10.1016/j.vaccine.2020.10.053. Epub 2020 Oct. 28. PMID: 33129609.
Zomber G, Reuveny S, Garti N, Shafferman A, Elhanany E. Effects of spontaneous deamidation on the cytotoxic activity of the Bacillus anthracis protective antigen. J Biol Chem. 2005 Dec. 2; 280(48):39897-906. doi: 10.1074/jbc.M508569200. Epub 2005 Sep. 27. PMID: 16188881.

While the preferred embodiments of the invention have been illustrated and described, it will be appreciated that various changes can be made therein without departing from the spirit and scope of the invention.

1. An immunogenic composition, comprising:
(a) 5 mcg to 100 mcg B. anthracis rPA (recombinant protective antigen) or mrPA (mutant rPA) protein; and
(b) liposome-embedded MPLA (monophosphoryl lipid A) adjuvant.

2. The immunogenic composition of claim 1, wherein the immunogenic composition comprises 5-15 mcg, 9-11 mcg, 45-55 mcg, or 75-85 mcg of rPA or mrPA protein.

3. The immunogenic composition of claim 1 or 2, wherein the immunogenic composition comprises 5 mcg to 200 mcg liposome-embedded MPLA adjuvant.

4. The immunogenic composition of claim 1 or 2, wherein the immunogenic composition comprises mrPA protein.

5. The immunogenic composition of claim 4, wherein the mrPA protein has the amino acid sequence of SEQ ID NO: 2.

6. An immunogenic composition, comprising:
60 mcg to 600 mcg B. anthracis mrPA (mutant rPA) protein,
wherein the composition is free of adjuvant.

7. The immunogenic composition of claim 6, wherein the immunogenic composition consists of:
60 mcg to 600 mcg B. anthracis mrPA (mutant rPA) protein; and
phosphate-buffered sucrose.

8. The immunogenic composition of claim 6 or 7, wherein the immunogenic composition comprises 70-90 mcg, 140-180 mcg or 220-260 mcg mrPA protein.

9. The immunogenic composition of claim 6 or 7, wherein the mrPA protein has the amino acid sequence of SEQ ID NO: 2.

10. A method for inducing an immune response to B. anthracis, comprising:
administering a first dose of the immunogenic composition, wherein the immunogenic composition comprises
(a) 5 mcg to 100 mcg B. anthracis rPA (recombinant protective antigen) or mrPA (mutant rPA) protein; and
(b) liposome-embedded MPLA (monophosphoryl lipid A) adjuvant.

11. The method of claim 10, further comprising:
administering a second dose of the immunogenic composition to the subject, wherein the first and second doses are administered 2-5 weeks apart.

12. The method of claim 10 or 11, wherein the administering is done by intramuscular injection.

13. The method of claim 10 or 11, wherein the first dose is administered to the subject after the subject is exposed to a source of B. anthracis spores.

14. The method of claim 10 or 11, wherein the method further comprises administering antibiotics to the subject, after the first dose.

15. The method of claim 14, wherein the antibiotics are administered to the subject 30-40 days after the first dose.

16. The immunogenic composition of claim 3, wherein the immunogenic composition comprises mrPA protein.

17. The immunogenic composition of claim 16, wherein the mrPA protein has the amino acid sequence of SEQ ID NO: 2.

18. The immunogenic composition of claim 8, wherein the mrPA protein has the amino acid sequence of SEQ ID NO: 2.

19. The method of claim 10 or 11, wherein the immunogenic composition comprises 5 mcg to 200 mcg liposome-embedded MPLA adjuvant.

20. The method of claim 10, wherein the immunogenic composition comprises mrPA protein.

21. The method of claim 20, wherein the mrPA protein has the amino acid sequence of SEQ ID NO: 2.

22. A method for inducing an immune response to B. anthracis, comprising: administering a first dose of the immunogenic composition of claim 6.

23. The method of claim 22, further comprising:
administering a second dose of the immunogenic composition to the subject, wherein the first and second doses are administered 2-5 weeks apart.

24. The method of claim 23, wherein the mrPA protein has the amino acid sequence of SEQ ID NO: 2.


SEQUENCE LISTING (5 sequences)

SEQ ID NO: 1 - protein, 735 aa, Bacillus anthracis
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 2 - protein, 733 aa, artificial sequence
  Description of Artificial Sequence Synthetic polypeptide
  mutated polypeptide
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSSNKESTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSETRTISKNTSTSRTHTSEVHGNADVHASDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 3 - protein, 4 aa, Bacillus anthracis
RKKR

SEQ ID NO: 4 - protein, 4 aa, artificial sequence
  Description of Artificial Sequence Synthetic peptide
SNKE

SEQ ID NO: 5 - protein, 4 aa, artificial sequence
  Description of Artificial Sequence Synthetic peptide
SNSS